(19) United States
(12) Patent Application Publication
(10) Pub. No.: US 2016/0325068 A1
(43) Pub. Date: Nov. 10, 2016

(54) MEASURING FLOW IN A RESPIRATORY THERAPY DEVICE

(71) Applicant: KONINKLIJKE PHILIPS N.V., Eindhoven (NL)

(72) Inventors: WILLIAM ANTHONY TRUSCHEL, OAKMONT, PA (US); BERNARD F. HETE, KITTANNING, PA (US)

(21) Appl. No.: 15/109,662

(22) PCT Filed: Dec. 30, 2014
(86) PCT No.: PCT/IB2014/067407

§ 371 Date: Jul. 5, 2016

Related U.S. Application Data
(60) Provisional application No. 61/923,860, filed on Jan. 6, 2014.

(51) Int. Cl. A61M 16/08; A61M 16/20; A61M 16/06; A61M 16/04; A61M 16/00; A61B 5/087

(52) U.S. Cl. CPC A61M 16/0866; A61M 16/0066; A61M 16/0875; A61B 5/087; A61M 16/0666; A61M 16/0465; A61M 16/0075; A61M 16/20; A61M 2016/0039; A61M 2230/40

(57) ABSTRACT

A system (10) for measuring subject flow in a respiratory therapy device. The system may include a primary pressure generator (12) connected to a subject circuit (18), and a secondary pressure generator (30) connected to a sensor circuit (36). The sensor circuit and subject circuit may be in fluid communication such that gas form the sensor circuit is introduced into the subject circuit. A measurement of flow parameters of gas through the sensor circuit may convey information related to flow parameters of gas in the subject circuit. The system may be configured such that contaminates from subject flow in the subject circuit do not flow into the sensor circuit.

### CROSS-REFERENCE TO RELATED APPLICATIONS

This patent application claims the priority benefit under 35 U.S.C. §119(e) from U.S. Provisional Application No. 61/923,860, filed on Jan. 6, 2014, the contents of which are herein incorporated by reference.

### BACKGROUND OF THE INVENTION

1. Field of the Invention

The present disclosure pertains to systems and methods of measuring subject flow in a respiratory therapy device.

2. Description of the Related Art

Respiratory therapy devices are configured to deliver a pressurized flow of breathable gas to the airway of a subject. During respiratory therapy, it may be desired to measure the flow parameters of gas related to inspiratory and expiratory flow phases of a respiratory system of the subject, referred to as subject flow. Such measurements may facilitate determining respiratory health problems of the subject, making adjustments to the pressurized flow of breathable gas delivered to the airway of the subject during therapy, and other operations. Subject flow may contain contaminates such as mucus, humidity and/or other particulates. These contaminates may interfere with the flow sensors used to measure the subject flow.

### SUMMARY OF THE INVENTION

Accordingly, one or more aspects of the present disclosure relate to a system for measuring subject flow in a respiratory therapy device. The system comprises a primary pressure generator configured to generate a first pressurized flow of breathable gas for delivery to a subject through a subject circuit. The subject circuit includes a first flow restriction. The first flow restriction causes a pressure difference to occur between a first side and a second side of the first flow restriction if gas is flowing through the subject circuit. The second side of the first flow restriction is opposite the first side of the first flow restriction. The system comprises a secondary pressure generator configured to generate a second pressurized flow of breathable gas for delivery to a sensor circuit. The sensor circuit is in fluid communication with the subject circuit such that gas from the second pressurized flow of breathable gas is introduced into the subject circuit. Gas is introduced into the subject circuit at a first interface and a second interface. The first interface is on the first side of the first flow restriction, and the second interface is on the second side of the first flow restriction. The system comprises a sensor in fluid communication with gas in the sensor circuit. The sensor is configured to generate output signals conveying information related to flow parameters of gas through the first flow restriction in the subject circuit.

Another aspect of the present disclosure relates to a method of measuring subject flow in a respiratory therapy device. The method comprises the operations of: providing a primary pressure generator configured to generate a first pressurized flow of breathable gas for delivery to a subject through a subject circuit, the subject circuit including a first flow restriction, the first flow restriction causing a pressure difference to occur between a first side and a second side of the first flow restriction if gas is flowing through the subject circuit, the second side being opposite the first side; providing a secondary pressure generator configured to generate a second pressurized flow of breathable gas for delivery to a sensor circuit; fluidly communicating gas from the second pressurized flow of breathable gas into the subject circuit at: a first interface on the first side of the first flow restriction, and a second interface on the second side of the first flow restriction; providing a sensor in fluid communication with gas in the sensor circuit, the sensor being configured to generate output signals conveying information related to flow parameters of gas through the first flow restriction in the subject circuit; and/or other operations.

Still another aspect of the present disclosure relates to a system for measuring subject flow in a respiratory therapy device. The system comprises primary flow generating means configured to generate a first pressurized flow of breathable gas for delivery to a subject through a subject circuit. The subject circuit includes a first flow restriction. The first flow restriction causes a pressure difference to occur between a first side and a second side of the first flow restriction if gas is flowing through the subject circuit. The second side of the first flow restriction is opposite the first side of the first flow restriction. The system comprises a secondary flow generating means configured to generate a second pressurized flow of breathable gas for delivery to a sensor circuit. The sensor circuit is in fluid communication with the subject circuit such that gas from the second pressurized flow of breathable gas is introduced into the subject circuit. Gas is introduced into the subject circuit at a first interface on the first side of the first flow restriction, and a second interface on the second side of the first flow restriction. The system comprises sensor means in fluid communication with gas in the sensor circuit. The sensor means is configured to generate output signals conveying information related to flow parameters of gas through the first flow restriction in the subject circuit.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 depicts an embodiment of the system for measuring subject flow in a respiratory therapy device comprising a subject circuit and a sensor circuit;

FIG. 2 depicts another embodiment of the system for measuring subject flow in a respiratory therapy device;

FIG. 3 shows an electrical equivalent system of the system of FIG. 1 used for analyzing the relationship between gas flow across the sensor employed in the sensor circuit of the system and subject flow in the subject circuit;

FIG. 4 is an illustration of a simulated plot depicting a linear relationship between gas flow across the sensor employed in the sensor circuit of the system and subject flow in the subject circuit based on the electrical equivalent system analysis;

FIG. 5 shows an illustration of a simulated plot depicting the flow of gas through the interfaces of the subject circuit and the sensor circuit across a range of subject flow based on the electrical equivalent system analysis;

FIG. 6 shows an experimentally obtained plot depicting a linear relationship between gas flow across the sensor employed in the sensor circuit of the system and subject flow in the subject circuit, showing similarity to the electrical equivalent system analysis;

FIG. 7 shows an experimentally obtained plot depicting the flow of gas through the interfaces of the subject circuit and the sensor circuit across a range of subject flow, showing similarity to the electrical equivalent system analysis;

FIG. 8 shows a view of a processor used in the system for measuring subject flow in a respiratory therapy device; and

FIG. 9 depicts a method of measuring subject flow in a respiratory therapy device.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

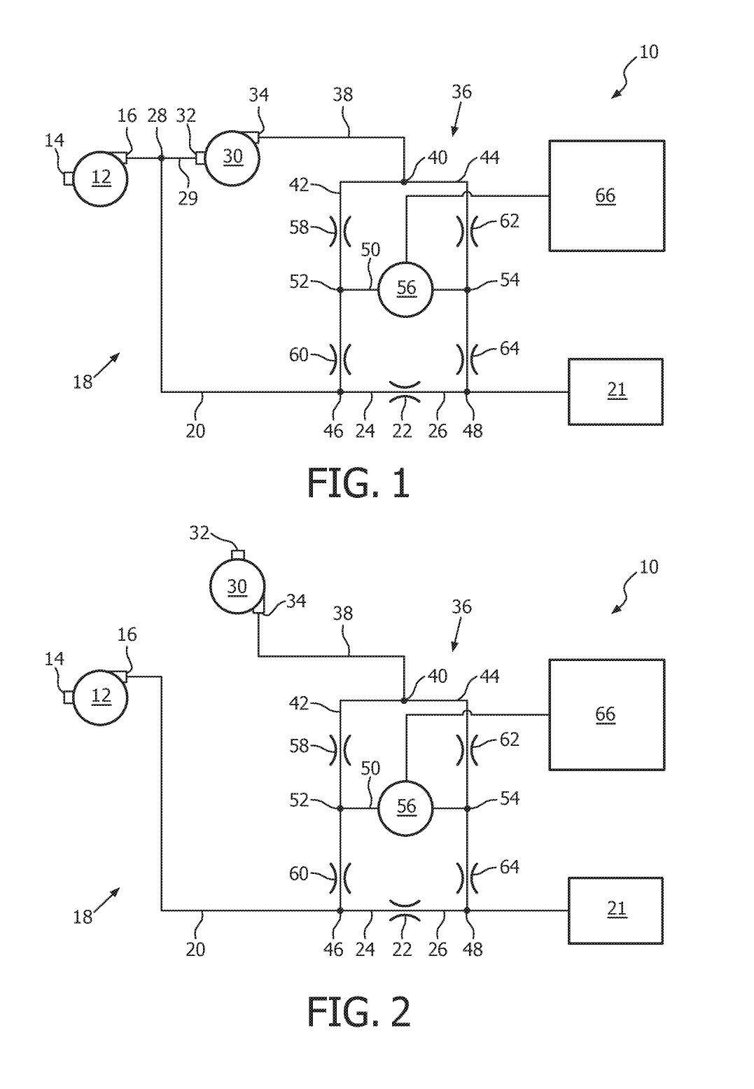

FIG. 1 schematically illustrates an exemplary embodiment of the system 10 for measuring subject flow (e.g., breathing parameters) in a respiratory therapy device. In some embodiments, system 10 comprises a primary pressure generator 12 connected to a subject circuit 18. Subject circuit 18 includes a primary conduit 20, a subject interface 21, and/or other components. System 10 comprises a secondary pressure generator 30 connected to a sensor circuit 36. Sensor circuit 36 includes a first conduit 38, a second conduit 42, a third conduit 44, a fourth conduit 50, one or more sensors 56, one or more processors 66, and/or other components.

In some embodiments, system 10 is configured to provide respiratory therapy to a subject. Primary pressure generator 12 is configured to generate a first pressurized flow of breathable gas for delivery to the airway of a subject through subject circuit 18 at subject interface 21. Subject interface 21 is configured to fluidly communicate with the airway of a subject. For example, subject interface 21 may include a nasal mask, nasal cannula, a full face mask, a nasal pillow mask, a hybrid mask, an oral mask, a total face mask, a tracheostomy tube, an endotracheal tube, and/or other invasive and/or non-invasive interface appliance configured to communicate a flow of pressurized gas within an airway of a subject. In some embodiments, subject interface 21 may be removably coupled to primary conduit 20. Subject interface 21 may be removed for cleaning and/or for other purposes.

Primary pressure generator 12 includes a fluid inlet 14 and a fluid outlet 16. Fluid inlet 14 may be in fluid communication with a gas source. A gas source may include, for example, one or more of the ambient environment, a gas reservoir, and/or other source of breathable gas. Primary pressure generator 12 may be any device, such as, for example, a pump, blower, piston, or bellows, that is capable of elevating the pressure of the received gas for delivery to a subject through subject circuit 18. Primary pressure generator 12 may comprise one or more valves for adjusting the delivery of the pressure/flow of gas. The present disclosure also contemplates controlling the operating speed of the blower, either alone or in combination with such valves, to control the pressure/flow of gas provided to the subject. Fluid outlet 16 of primary pressure generator 12 is in fluid communication with primary conduit 20. Primary conduit 20 may be one or more of a flexible conduit, sealed tubing, and/or other component suitable for delivery of pressurized breathable gas.

Primary pressure generator 12 is configured to generate a first pressurized flow of breathable gas according to one or more parameters of the flow of gas (e.g., flow rate, pressure, volume, temperature, gas composition, etc.) for therapeutic purposes, and/or for other purposes. By way of a non-limiting example, primary pressure generator 12 is configured to generate the first pressurized flow at a flow rate and/or pressure of the flow of gas to provide mechanical ventilation to the airway of a subject. For example, mechanical ventilation in the form of pressure control ventilation may require the first pressurized flow of breathable gas to be delivered to the subject in a pressure range generally of 0 to 30 cmH₂O at a flow rate of 0 to 120 liters per minute (lpm) for any given time interval (e.g., during inspiratory and expiratory breath phases of the subject). However, in some embodiments, primary pressure generator 12 is configured to provide respiratory therapy in accordance with other therapeutic purposes, for example continuous positive airway pressure (CPAP), high flow therapy, volume-controlled therapy, and/or other respiratory therapy. Other therapy regimes may require different flow/pressure parameters of the first pressurized flow of breathable gas delivered by primary pressure generator 12.

In some embodiments it is envisioned that some or all of the features of system 10 can be adapted to for employment (e.g., via retrofitting) with existing respiratory therapy devices, such as a CPAP device and/or mechanical ventilation device. For example, it is envisioned that sensor circuit 36 and/or other components may be retrofitted to an existing respiratory therapy device to perform the various features and functions of the embodiments described herein.

Subject circuit 18 includes a first flow restriction 22. First flow restriction 22 may be a calibrated fixed orifice and/or a flow resistive element. For example, first flow restriction 22 may be a needle valve and/or other suitable flow restrictive element included in primary conduit 20. In some embodiments, first flow restriction 22 may be retrofitted into a gas flow circuit of an existing respiratory therapy device to facilitate measuring subject flow as described herein.

First flow restriction 22 includes a first side 24. First side 24 is a portion of primary conduit 20 proximal to first flow restriction 22 which is adjacent to primary pressure generator 12. First flow restriction 22 includes a second side 26 opposite first side 24. Second side 26 is a portion of primary conduit 20 proximal to first flow restriction 22 which is adjacent to subject interface 21. First flow restriction 22 is calibrated such that a flow rate of pressurized gas through first flow restriction 22 (e.g., through subject circuit 18) may be calculable given a pressure difference across first flow restriction 22, and vice versa. For example a pressure difference may occur between first side 24 and a second side 26 of the first flow restriction 22 if gas is flowing through the subject circuit 18 at some flow rate.

Secondary pressure generator 30 is configured to generate a second pressurized flow of breathable gas for delivery to sensor circuit 36. Secondary pressure generator 30 is configured to generate the second pressurized flow of breathable gas according to one or more parameters of the flow of gas (e.g., flow rate, pressure, volume, temperature, gas composition, etc.). Secondary pressure generator 30 includes a fluid inlet 32 and a fluid outlet 34. Fluid inlet 32 is in fluid communication with a gas source. In FIG. 1, fluid inlet 32 is in fluid communication with fluid outlet 16 of primary pressure generator 12 such that gas from the first pressurized flow of breathable gas delivered by primary pressure generator 12 is the gas source for secondary pressure generator 30. For example, the first pressurized flow of breathable gas delivered by primary pressure generator 12 into primary conduit 20 may split 28 to a conduit portion 29 (e.g., via a connector or the like) in fluid communication with fluid inlet 32 of secondary pressure generator 30.

The remainder of primary conduit 20 after split 28 includes first flow restriction 22 and subject interface 21 components engaged to primary conduit 20. Therefore a portion of the first pressurized flow of breathable gas may be delivered to fluid inlet 32 of secondary pressure generator 30. In some embodiments, secondary pressure generator 30 generates the second pressurized flow of breathable gas which is higher in pressure than the first pressurized flow of breathable gas.

Secondary pressure generator 30 may be any device, such as, for example, a pump, blower, piston, or bellows, that is capable of elevating the pressure of the received first pressurized flow of breathable gas for delivery to sensor circuit 36 to the pressure of the second pressurized flow. Secondary pressure generator 30 may comprise one or more valves for adjusting the delivery of the pressure/flow of gas. The present disclosure also contemplates controlling the operating speed of the blower, either alone or in combination with such valves, to control the pressure/flow of gas delivered to sensor circuit 36.

In the embodiment of system 10 shown in FIG. 1 and/or other embodiments, the pressure elevation requirements of secondary pressure generator 30 are relatively low (e.g., compared to primary pressure generator 12) since the gas from the first pressurized flow delivered to fluid inlet 32 of secondary pressure generator 30 has already be elevated to a certain pressure (e.g., the pressure of the first pressurized flow delivered by primary pressure generator 12). For example, primary pressure generator 12 may generate the first pressurized flow at a pressure of 25 cmH₂O for a time period, such that gas introduced into secondary pressure generator 30 has the same or similar pressure during that time period. It may be a requirement of system 10 to deliver the second pressurized flow of breathable gas to sensor circuit 36 at a higher pressure, for example, 30 cmH₂O. Thus secondary pressure generator 30 may only need to elevate the pressure a fraction higher than the pressure of the gas being introduced into second pressure generator 30, and can be accomplished at a constant low speed with minimal power requirements. This configuration is less likely to disturb the flow/pressure parameters of the first pressurized flow of breathable gas associated with the therapy regime delivered by primary pressure generator 12.

As shown in FIG. 2, in some embodiments, system 10 is configured such that fluid inlet 32 of secondary pressure generator 30 is in fluid communication with a gas reservoir, ambient environment, and/or other suitable source of breathable gas. In this embodiment, system 10 is configured such that sensor circuit 36 does not receive gas from the first pressurized flow delivered from primary pressure generator 12. The second pressured flow delivered by secondary pressure generator 30 into sensor circuit 36 may comprise gas which is provided solely from secondary pressure generator 30 (e.g., absent gas from first pressurized flow). This embodiment may require a higher gas pressure/flow elevation to be performed by secondary pressure generator 30 in order to obtain the pressure/flow parameters of the second pressurized flow of breathable gas delivered to sensor circuit 36 (e.g., compared to the pressure of ambient environment gas, for example). This embodiment may also require secondary pressure generator 30 to continually adjust the pressure delivered into sensor circuit 36 during operation (e.g., to match changes in pressure of the flow of gas delivered by primary pressure generator 12).

Returning to FIG. 1, sensor circuit 36 is in fluid communication with the subject circuit 18 such that gas from the second pressurized flow of breathable gas is introduced into subject circuit 18 at one or more interfaces. The interfaces may create high pressure nodes within subject circuit 18 such that gas tends to flow into subject circuit 18 from sensor circuit 36. For example, the second pressurized flow of breathable gas delivered by secondary pressure generator 30 is introduced into subject circuit 18 at a first interface 46 and a second interface 48. First interface 46 is located on first side 24 of first flow restriction 22. Second interface is located on second side 26 of first flow restriction 22. First interface 46 and second interface 48 may include one or more of a connector, a valve, a fitting, a bonded plastic tube, T-fitting, and/or other suitable interface for fluidly connecting sensor circuit 36 to subject circuit 18 as described herein.

The present disclosure contemplates that system 10 is used to measure subject flow of gas within subject circuit 18 via sensor circuit 36, while preventing any gas or particles within subject circuit 18 from contaminating sensor circuit 36. In particular, system 10 maintains one or more high pressure nodes at the one or more interfaces where subject circuit 18 fluidly connects with sensor circuit 36. The high pressure nodes at the interfaces are provided by maintaining a relatively higher gas pressure in sensor circuit 36 than in subject circuit 18 over the range of subject flows to be measured. The system 10 here may be configured such that no retrograde subject flow contaminates sensor circuit 36, and in particular does not contaminate sensor 56 connected to sensor circuit 36.

Sensor circuit 36 comprises first conduit 38 in fluid communication with fluid outlet 34 of secondary pressure generator 30. First conduit 38 includes split 40 (e.g., via a connector or the like) to divide the second pressurized flow of breathable gas delivered by secondary pressure generator 30 into circuit legs including second conduit 42 and third conduit 44. Second conduit 42 includes an outlet which fluidly interfaces subject circuit 18 at first interface 46. Third conduit 44 includes an outlet which fluidly interfaces subject circuit 18 at second interface 48.

System 10 is configured such that sensor circuit 36 includes a fourth conduit 50 in fluid communication with gas in sensor circuit 36. Fourth conduit 50 is in fluid communication at one end with second conduit 42 at a third fluid interface 52. Third interface 52 is between split 40 and first interface 46. Fourth conduit 50 is in fluid communication at an opposite end with third conduit 44 at a fourth interface 54. Fourth interface 54 is between split 40 and second interface 48. System 10 herein additionally includes sensor 56 in fluid communication with gas in sensor circuit 36. Sensor 56 may be fluidly coupled to fourth conduit 50 to facilitate the fluid communication of gas in sensor circuit 36 with sensor 56.

System 10 herein is configured to eliminate and/or minimize gas flow from subject circuit 18 into sensor circuit 36 through first interface 46 and second interface 48 due to, at least in part, the relatively higher pressure second pressurized flow of breathable gas delivered by secondary pressure generator 30 compared to the relatively lower pressure first pressurized flow of breathable gas delivered into subject circuit 18 by primary pressure generator 12. System 10 is configured such that a predictable relationship between subject flow within subject circuit 18 and the flow of gas through sensor circuit 36, or more specifically, through sensor 56 (e.g., sensor flow) exists when subject interface 21 is connected to the airway of the subject during respiratory therapy and gas is flowing through system 10. Such a relationship may allow a measurement of flow parameters of the flow of gas within sensor circuit 36 to convey information related to flow parameters of gas through subject circuit 18. As such, direct contact of one or more flow sensors with the flow of gas in subject circuit 18 is not needed for such measurements. This will reduce, if not eliminate, the chance of particulate contamination from gas in subject circuit 18 (e.g., mucus from subject flow) from contaminating the sensor employed in sensor circuit 36, such as sensor 56.

In some embodiments, sensor circuit 36 may include one or more calibrated orifices and/or flow restrictive elements. The application of such flow restrictive elements into sensor circuit 36 may allow a user to calibrate and/or further understand predictive relationships of flow parameters of gas flowing across first flow restriction 22 (e.g., subject flow) and gas through fourth conduit 50 (e.g., sensor flow).

In some embodiments, second conduit 42 may comprise of a second flow restriction 58 between split 40 of first conduit 38 and third interface 52. Second conduit 42 may include a third flow restriction 60 between third interface 52 and first interface 46. Third conduit 44 may comprise a fourth flow restriction 62 between split 40 of first conduit 38 and fourth interface 54. Third conduit 44 may include a fifth flow restriction 64 between fourth interface 54 and second interface 48. In some embodiments second flow restriction 58, third flow restriction 60, fourth flow restriction 62, and fifth flow restriction 64 may comprise one or more of a needle valve, a calibrated orifice having a known diameter and length, a length or section of conduit of known diameter and length (e.g., wherein resistance to flow is provided by the inherent drag on the conduit walls), and/or other flow restrictive element suitable for the intended purpose.

Sensor 56 and first flow restriction 22 are connected in parallel with respect to second conduit 42 and third conduit 44 such that a relationship between flow across sensor 56 (e.g., sensor flow) and flow across first flow restriction 22 (e.g., subject flow) is established within system 10 when gas is flowing through system 10. Sensor 56 is configured to generate output signals relating to the flow parameters of gas through fourth conduit 50. System 10 is configured such that the flow parameters of gas through fourth conduit 50 convey information related to flow parameters of gas through first flow restriction 22 in subject circuit 18. How parameters of gas through first flow restriction 22 may correspond to subject flow within subject circuit 18 when subject interface 21 is connected to the airway of the subject and gas is flowing through system 10. Thus a measure of flow parameters of gas through fourth conduit 50 (e.g., across sensor 56) may provide information related to subject flow through subject circuit 18.

In some embodiments, sensor 56 may include a differential pressure sensor, mass flow anemometer, and/or other sensor configured to generate output signals relating to flow parameters of gas through sensor circuit 36 (e.g., fourth conduit 50). For example sensor 56 may measure the flow rate of gas through fourth conduit 50 either directly or indirectly. As mentioned, system 10 herein is configured such that a relationship between the flow parameters of gas across sensor 56 and gas across first flow restriction 22 is established and/or are calculable. For example, calibrating first flow restriction 22, second flow restriction 58, third flow restriction 60, fourth flow restriction 62 and fifth flow restriction 64 to known values, a linear relationship between flow across sensor 56 and flow across first flow restriction 22 may be established (described in more detail herein). In some embodiments, the respective calibrations of second flow restriction 58 and fourth flow restriction 62 in system 10 may effectively establish the relationship between the flow of gas across first flow restriction 22 and sensor 56. The respective calibrations of third flow restriction 60 and fifth flow restriction 64 may be tuned to limit the flow through the first interface 46 and second interface 48.

In some embodiments, system 10 may comprise one or more processing units, such as processor 66 connected to sensor 56. Processor 66 is configured to execute computer program components to determine subject flow from output signals generated by sensor 56. Referring now to FIG. 8, processor 66 may include electronic storage 72 and computer program components comprising a sensor flow component 68 and a subject flow component 70. Sensor flow component 68 is configured to determine, from the output signals generated by sensor 56, flow parameters such as flow rate, of the flow of breathable gas passing through fourth conduit 50 of the sensor circuit 36 and across sensor 56. Subject flow component 70 is configured to determine flow parameters of the flow of breathable gas through the first flow restriction 22 of the subject circuit 18. The determination may be based on the relationship of the flow parameters of the flow of gas through fourth conduit 50 of sensor circuit 36 and the flow parameters of the flow of gas through first flow restriction 22 of subject circuit 18. The relationship may be a linear relationship as determined from analysis described below.

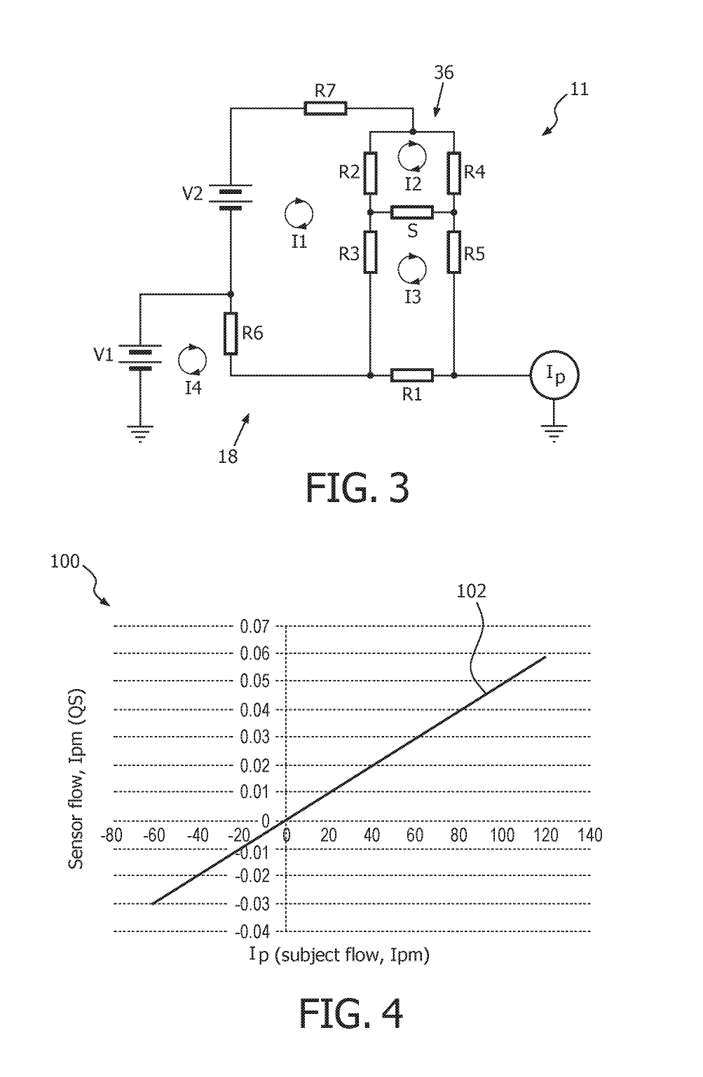

A solution of determining the relationship between flow parameters of the flow of gas through fourth conduit 50 of sensor circuit 36 (e.g., sensor flow) and the flow parameters of the flow of gas through first flow restriction 22 of subject circuit 18 (e.g., subject flow) is through analysis of an electrical equivalent system 11 as shown in FIG. 3. Briefly, fluid flow through conduit systems is typically analogous to electrical current flow through electrical circuits. In electrical equivalent system 11, various components of system 10 will be represented by electrical equivalents, and the analysis using conventional electrical system analysis techniques will provide information related to fluid flow through the system 10.

Referring now to both system 10 in FIG. 1 and electrical equivalent system 11 in FIG. 3, in electrical equivalent system 11, the pressure sources (e.g., primary pressure generator 12 and secondary pressure generator 30 of system 10) are represented by ideal voltage sources, V1 and V2 respectively. The restrictive elements and conduits are represented by linear resistors, corresponding to the resistance to fluid flow through system 10 (e.g., drag produced by conduit walls). It is noted that a similar electrical equivalent system may be developed and analyzed to simulate flow through the embodiment of system 10 shown in FIG. 2.

For example, first flow restriction 22 of system 10 is represented by resistor R1 in electrical equivalent system 11. Second flow restriction 58 is represented by resistor R2. Third flow restriction 60 is represented by resistor R3. Fourth flow restriction 62 is represented by resistor R4. Fifth flow restriction 64 is represented by resistor R5. Primary conduit 20 of subject circuit 18 is represented by resistor R6. First conduit 38 is represented by resistor R7. Subject flow is represented by current Ip, which is the sum of the flow through R1 and R5. Sensor 56 is represented by resistor Rs (e.g., for embodiments of system 10 where sensor 56 is of the hot wire anemometer type sensor). In some embodiments, if a differential pressure sensor is employed for sensor 56, the flow through fourth conduit 50 would be an open circuit in electrical equivalent system 11. In addition, the various current flows I1-I4 can be used to represent the directional flow rates of fluid through the respective system parts as shown. For example the flow through R1 (e.g., first flow restriction 22) is I3-I4, and the flow through R5 (e.g., through second interface 48) is I3.

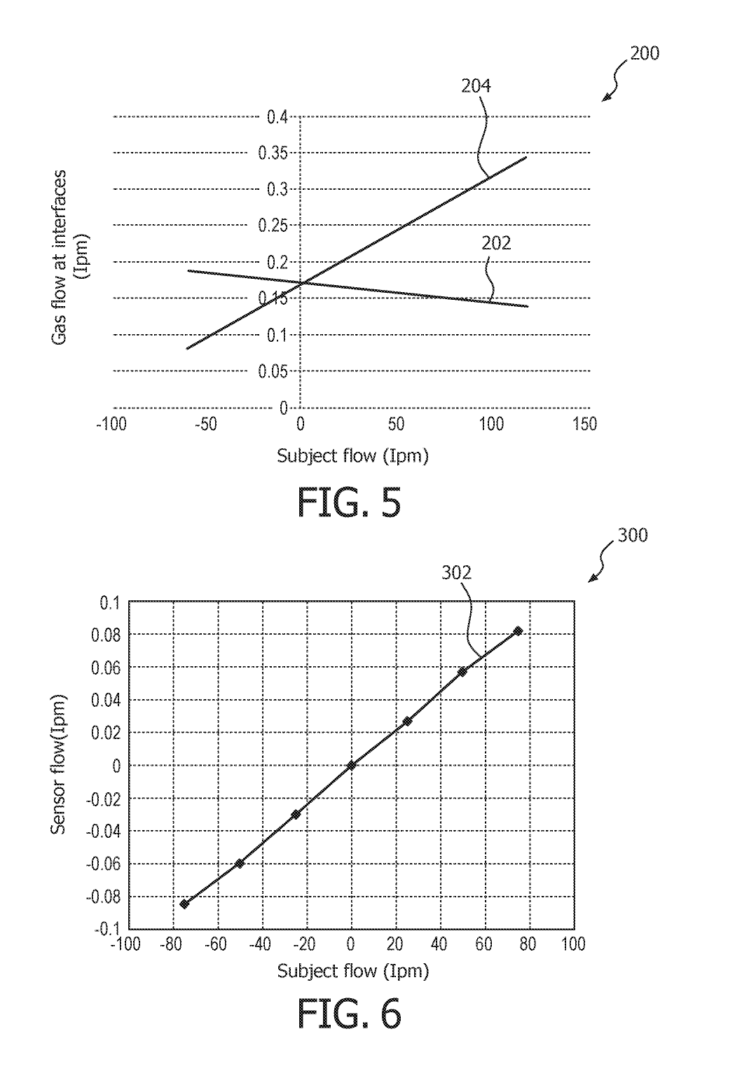

The design constraints employed for analysis of electrical equivalent system 11 are 1) the flow through R3 (herein “Q3”) and the flow through R5 (herein “Q5) is always greater than zero (e.g., flowing from sensor circuit 36 through first interface 46 and second interface 48 into subject circuit 18 only) and 2) that the flow through S (herein QS) is some predictable function of Ip. The first constraint represents the elimination of retrograde flow of gas into sensor circuit 36 from subject circuit 18 as outlined previously. In addition, using conventional electrical system analysis techniques, we know that the flow through R3 is equal to I1-I3 (e.g., Q3=I1-I3), and that the flow through R5 is I3 (e.g., Q5=I3). Solutions to the electrical equivalent system 11 that include linear approximations for standard and/or known tubing and restrictive components in the fluidic system 10 including a known mass flow sensor in place of S, predict the following relationships illustrated in FIG. 4 and FIG. 5.

FIG. 4 depicts a simulated plot 100 showing a linear relationship 102 between sensor flow (QS) and subject flow (Ip), for reasonable values of subject flow (e.g., approximately −60 to 120 lpm). Flow in the figure is represented in liters per minute (lpm). Using plot 100 and/or data making up plot 100, subject flow can be extrapolated when flow across sensor 56 is known (e.g., measured). For the present simulation, the linear relationship concluded that subject flow is approximately 2000 times greater than the flow measured at sensor 56.

As mentioned previously, it is the intent of the present disclosure to provide a system 10 which prevents contaminants from subject flow (e.g., mucus, humidity, etc.) from contaminating the sensor within sensor circuit 36. Referring now to FIG. 5, a simulated plot 200 shows simulated data based on the electrical equivalent system analysis depicting the gas flow at the interfaces (y-axis) of subject circuit 18 and sensor circuit 36 (e.g., at first interface 46 and second interface 48 in FIG. 1) with respect to subject flow in subject circuit (x-axis). In this plot, a positive flow rate on the y-axis refers to flow in the direction across the interfaces from sensor circuit 36 to subject circuit 18, therefore meeting the first constraint. A first line 202 depicts data representing the flow of gas through first interface 46 (e.g., flow Q3 through R3) over a range of subject flow. A second line 204 depicts data representing the flow of gas through second interface 48 (e.g., flow Q5 through R5) over a range of subject flow (e.g., flow rate in liters per minute). For example, over a range of subject flow (x-axis) of about −60 lpm to 120 lpm, it can be seen that the flow of gas through both interfaces remains positive (e.g., remains above the 0 flow rate on the y-axis), therefore all retrograde flow from the subject circuit is eliminated.

FIG. 6 depicts a plot 300 showing experimental results employing system 10, used to confirm the simulated results discussed above. The experimental results shown confirm the relationship established in the simulated results with differences only pertaining to the linearity of the waveforms. These differences were expected from the linear approximations that were made in the simulation. In experimental analysis of system 10, subject interface 21 is replaced by a blower or other pressure generator configured to draw gas through subject circuit 18 in accordance with reasonable subject flow (e.g., approximately −60 to 120 lpm). First flow restriction 22 is calibrated to 10 cmH20 at 120 lpm. Second flow restriction 58, third flow restriction 60, fourth flow restriction 62 and fifth flow restriction 64 are calibrated to provide a 5cmH₂O pressure drop across sensor 56 when flow is delivered through first flow restriction 22 at 120 lpm. Primary conduit 20 is a 6 foot smooth bore patient circuit conduit. Various flow measuring devices are employed throughout system 10 to measure flow parameters while gas is flowing through both sensor circuit 36 and subject circuit 18. In some embodiments, system 10 was calibrated such that second flow restriction 58 and fourth flow restriction 62 were calibrated as identically as possible. In some embodiments system 10 was calibrated such that third flow restriction 60 and fifth flow restriction 64 were calibrated identically as possible.

Subject flow may be extrapolated from plot 300 for a given measured sensor flow. The determined subject flow may then be displayed to a user, and/or used for other purposes, such as calibration and/or adjustment of the pressurized flow of breathable gas delivered by one or both of primary pressure generator 12 and secondary pressure generator 30. It is noted that flow of gas across sensor 56 is considered positive when flowing from third interface 52 to fourth interface 54, and negative for flow in the opposite direction (see, e.g., FIG. 1). It is also noted that flow of gas within subject circuit 18 (e.g., subject flow) is considered positive when flowing from primary pressure generator 12 towards subject interface 21 (e.g., subject is inhaling), and negative for flow in the opposite direction, e.g., exhalation (see, e.g., FIG. 1).

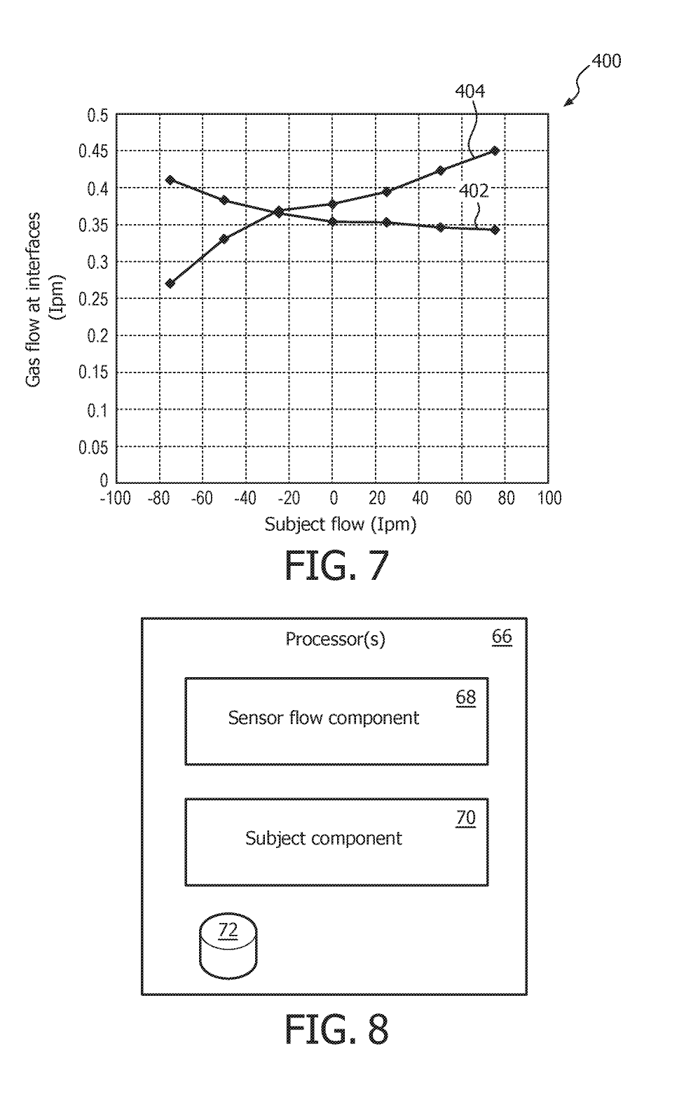

FIG. 7 shows a plot 400 depicting experimental results which concludes similar results as shown in the simulated plot of FIG. 5. A first line 402 depicts data representing the flow of gas through first interface 46 over a range of subject flow. A second line 404 depicts data representing the flow of gas through second interface 48 over a range of subject flow (e.g., flow rate in liters per minute). Flow is shown remaining positive (e.g., above the “0” on the y-axis) for the entire range of subject flow.

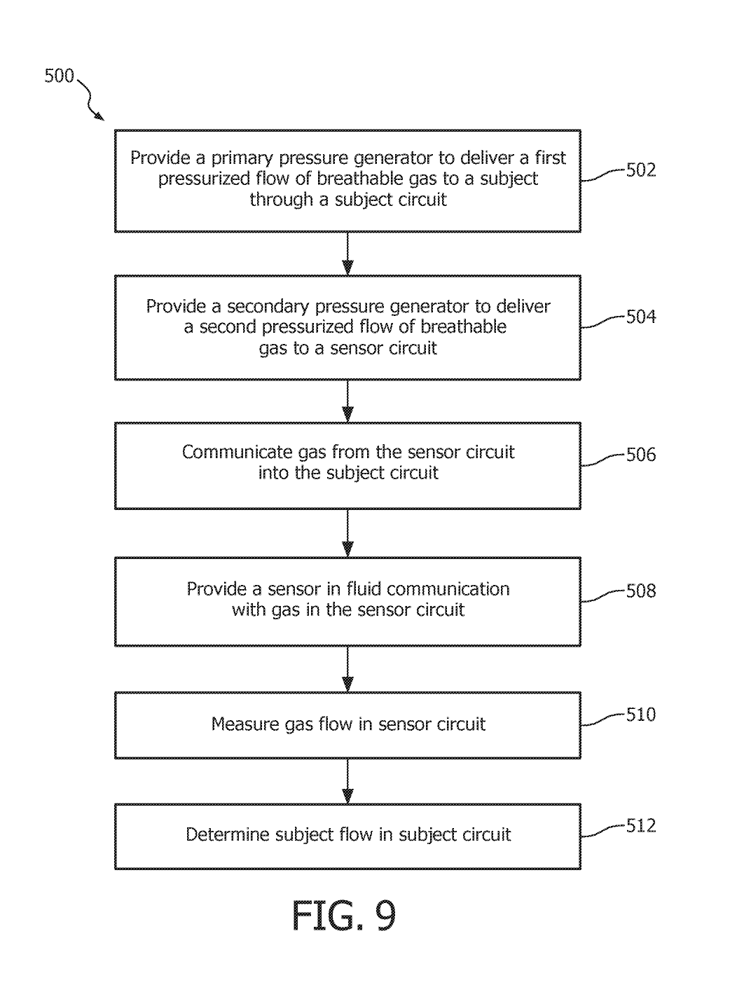

FIG. 9 illustrates a method 500 of measuring subject flow in a respiratory therapy device. The operations of method 500 presented below are intended to be illustrative. In some embodiments, method 500 may be accomplished with one or more additional operations not described, and/or without one or more of the operations discussed. Additionally, the order in which the operations of method 500 are illustrated in FIG. 9 and described below is not intended to be limiting.

At an operation 502, a primary pressure generator may be provided. The primary pressure generator may be configured to deliver a first pressurized flow of breathable gas to the airway of a subject through a subject circuit. In some embodiments, operation 502 may be performed with a primary pressure generator and subject circuit the same or similar as primary pressure generator 12 and subject circuit 18 (shown in FIGS. 1 and 2 and described herein).

At an operation 504, a secondary pressure generator may be provided. The secondary pressure generator may be configured to deliver a second pressurized flow of breathable gas to a sensor circuit. In some embodiments, operation 504 may be performed with a secondary pressure generator and sensor circuit the same or similar as secondary pressure generator 30 and sensor circuit 36 (shown in FIGS. 1 and 2 and described herein).

At an operation 506, a gas may be communicated from the sensor circuit to the subject circuit. In some embodiments, operation 506 is performed with one or more interfaces of the subject circuit and the sensor circuit the same or similar as first interface 46 and second interfaces 48 (shown in FIGS. 1 and 2 and described herein).

At an operation 508, a sensor in fluid communication with gas in the sensor circuit may be provided. In some embodiments, operation 508 may be performed by a sensor the same or similar to sensor 56 (shown in FIGS. 1 and 2 and described herein).

At an operation 510 the flow of gas within sensor circuit may be measured. In some embodiments, operation 510 may be performed by a sensor the same or similar to sensor 56 (shown in FIGS. 1 and 2 and described herein).

At an operation 512, the flow of gas within the subject circuit (e.g., subject flow) may be determined. In some embodiments, operation 512 may be performed by one or more processors the same or similar as processor 66 (shown in FIGS. 1, 2 and 8 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system for measuring subject flow in a respiratory therapy device, the system comprising:
a primary pressure generator configured to generate a first pressurized flow of breathable gas for delivery to a subject through a subject circuit, the subject circuit including a first flow restriction, the first flow restriction causing a pressure difference to occur between a first side and a second of the first flow restriction if gas is flowing through the subject circuit, the second side being opposite the first side;
a secondary pressure generator configured to generate a second pressurized flow of breathable gas for delivery to a sensor circuit, the sensor circuit being in fluid communication with the subject circuit such that gas from the second pressurized flow of breathable gas is introduced into the subject circuit at:
a first interface on the first side of the first flow restriction, and
a second interface on the second side of the first flow restriction; and
a sensor in fluid communication with gas in the sensor circuit, the sensor being configured to generate output signals conveying information related to flow parameters of gas through the first flow restriction in the subject circuit.

2. The system of claim 1, wherein the sensor circuit comprises a first conduit (38) connected to the secondary pressure generator which splits to divide the second pressurized flow into a second conduit and a third conduit, the second conduit having an outlet which fluidly interfaces the subject circuit at the first interface, and the third conduit having an outlet which fluidly interfaces the subject circuit at the second interface.

3. The system of claim 2, wherein the sensor circuit additionally includes a fourth conduit in fluid communication with the second pressurized flow of breathable gas, the fourth conduit being in fluid communication with the second conduit at a third interface and the third conduit at a fourth interface, and wherein the sensor is coupled to the fourth conduit to facilitate the fluid communication of the sensor with gas in the sensor circuit, such that the sensor and first flow restriction are connected in parallel with respect to the second conduit and the third conduit of the sensor circuit.

4. The system of claim 3, wherein the second conduit additionally comprises a second flow restriction between the split of the first conduit and the third interface, and a third flow restriction between the third interface and the first interface, and the third conduit additionally comprises a fourth flow restriction between the split of the first conduit and fourth interface, and a fifth flow restriction between the fourth interface and the second interface.

5. The system of claim 1, further comprising one or more processors configured to execute computer program components, the computer program components comprising:
a sensor flow component configured to determine, from the output signals generated by the sensor, flow parameters of the flow of breathable gas passing through the fourth conduit of the sensor circuit; and
a subject flow component configured to determine flow parameters of the flow of breathable gas through the first flow restriction of the subject circuit, the determination based on a linear relationship of the flow parameters of the flow of gas through the fourth conduit of the sensor circuit and the flow parameters of the flow of gas through the first flow restriction of the subject circuit.

6. A method of measuring subject flow in a respiratory therapy device, the method comprising:
providing a primary pressure generator configured to generate a first pressurized flow of breathable gas for delivery to a subject through a subject circuit, the subject circuit including a first flow restriction, the first flow restriction causing a pressure difference to occur between a first side and a second side of the first flow restriction if gas is flowing through the subject circuit, the second side being opposite the first side;
providing a secondary pressure generator configured to generate a second pressurized flow of breathable gas for delivery to a sensor circuit;
fluidly communicating gas from the second pressurized flow of breathable gas into the subject circuit at:
a first interface on the first side of the first flow restriction, and
a second interface on the second side of the first flow restriction; and
providing a sensor in fluid communication with gas in the sensor circuit, the sensor being configured to generate output signals conveying information related to flow parameters of gas through the first flow restriction in the subject circuit.

7. The method of claim 6, wherein the sensor circuit comprise a first conduit connected to the secondary pressure generator which splits to divide the second pressurized flow into a second conduit and a third conduit, the second conduit having an outlet which fluidly interfaces the subject circuit at the first interface, and the third conduit having an outlet which fluidly interfaces the subject circuit at the second interface.

8. The method of claim 6, wherein the sensor circuit additionally includes a fourth conduit in fluid communication with the second pressurized flow of breathable gas, the fourth conduit being in fluid communication with the second conduit at a third interface and the third conduit at a fourth interface, and wherein the sensor is coupled to the fourth conduit to facilitate the fluid communication of the sensor with gas in the sensor circuit, such that the sensor and first flow restriction are connected in parallel with respect to the second conduit and the third conduit of the sensor circuit.

9. The method of claim 6, wherein the second conduit additionally comprises a second flow restriction between the split of the first conduit and the third interface, and a third flow restriction between the third interface and the first interface, and the third conduit additionally comprises a fourth flow restriction between the split of the first conduit and fourth interface, and a fifth flow restriction between the fourth interface and the second interface.

10. The method of claim 6, further comprising:
performing, on one or more physical processors and storage media storing machine-readable instructions, the operations of:
determining, from the output signals generated by the sensor, flow parameters of the flow of breathable gas passing through the fourth conduit of the sensor circuit; and
determining flow parameters of the flow of breathable gas through the first flow restriction of the subject circuit, the determination based on a linear relationship of the flow parameters of the flow of gas through the fourth conduit of the sensor circuit and the flow parameters of the flow of gas through the first flow restriction of the subject circuit.

11. A system for measuring subject flow in a respiratory therapy device, the system comprising:
primary flow generating means configured to generate a first pressurized flow of breathable gas for delivery to a subject through a subject circuit, the subject circuit including a first flow restriction, the first flow restriction causing a pressure difference to occur between a first side and a second side of the first flow restriction if gas is flowing through the subject circuit, the second side being opposite the first side;
secondary flow generating means configured to generate a second pressurized flow of breathable gas for delivery to a sensor circuit, the sensor circuit being in fluid communication with the subject circuit such that gas from the second pressurized flow of breathable gas is introduced into the subject circuit at:
a first interface on the first side of the first flow restriction, and
a second interface on the second side of the first flow restriction; and
sensor means in fluid communication with gas in the sensor circuit, the sensor means being configured to generate output signals conveying information related to flow parameters of gas through the first flow restriction in the subject circuit.

12. The system of claim 11, wherein the sensor circuit comprise a first conduit to the secondary flow generating means which splits to divide the second pressurized flow into a second conduit and a third conduit, the second conduit having an outlet which fluidly interfaces the subject circuit at the first interface, and the third conduit having an outlet which fluidly interfaces the subject circuit at the second interface.

13. The system of claim 12, wherein the sensor circuit additionally includes a fourth conduit in fluid communication with the second pressurized flow of breathable gas, the fourth conduit being in fluid communication with the second conduit at a third interface and the third conduit at a fourth interface, and
wherein the sensing means is coupled to the fourth conduit to facilitate the fluid communication of the sensor with gas in the sensor circuit, such that the sensing means and first flow restriction are connected in parallel with respect to the second conduit and the third conduit of the sensor circuit.

14. The system of claim 13, wherein:
the second conduit additionally comprises a second flow restriction between the split of the first conduit and the third interface, and a third flow restriction between the third interface and the first interface, and
the third conduit additionally comprises a fourth flow restriction between the split of the first conduit and fourth interface, and a fifth flow restriction between the fourth interface and the second interface.

15. The system of claim 11, additionally comprising one or more processing means configured to execute computer program components, the computer program components comprising:
a sensor flow component configured to determine, from the output signals generated by the sensor, flow parameters of the flow of breathable gas passing through the fourth conduit of the sensor circuit; and
a subject flow component configured to determine flow parameters of the flow of breathable gas through the first flow restriction of the subject circuit, the determination based on a linear relationship of the flow parameters of the flow of gas through the fourth conduit of the sensor circuit and the flow parameters of the flow of gas through the first flow restriction of the subject circuit.
